(19) United States
(12) Patent Application Publication
(10) Pub. No.: US 2006/0067948 A1
(43) Pub. Date: Mar. 30, 2006

(54) Viral vectors

(76) Inventors: Jane Frances Allen, Hertfordshire (GB); Stephen Daniel Charles Griffin, Leeds the UK (GB); Andrew Michael Lindsey Lever, Cambridge the UK (GB)

Correspondence Address: NIXON & VANDERHYE, PC, 901 NORTH GLEBE ROAD, 11TH FLOOR, ARLINGTON, VA, 22203, US

(21) Appl. No.: 10/473,412

(22) PCT Filed: Mar. 28, 2002
(86) PCT No.: PCT/GB02/01518

§ 371 Date: Dec. 12, 2005

(30) Foreign Application Priority Data
Mar. 30, 2001 (GB) 0108065.4

(51) Int. Cl. A61K 39/21; C12N 7/00; C12N 15/867

(52) U.S. Cl. 424/208.1; 435/456; 435/235.1

(57) ABSTRACT

A Human Immunodeficiency Virus (HIV-2) vector having a mutation within a packaging signal such that viral RNA is not packaged within an HIV-2 capsid is described. A further vector comprises an HIV-2 packaging signal and a heterologous gene capable of being expressed in the vector. These vectors may be co-transfected into a host cell to produce HIV-2 virus particles capable of expressing a heterologous gene.

### FIELD OF THE INVENTION

This invention relates to vectors and their use in gene transfer. The vectors are based on retroviruses, adapted so that they cannot package their own RNA, and which can be used as infectious agents to transfer foreign genes, e.g. for somatic gene therapy.

### BACKGROUND OF THE INVENTION

Retroviruses are classified in several ways. They are divided into various groups on the basis of their morphology. These groups are A, B. C and D type viruses. They are also classified as belonging to one of three subfamilies, namely oncoviruses, spumaviruses and lentiviruses.

The family of retroviruses designated C-type viruses are characterised by capsid assembly at the cell membrane, and include viruses of the lentivirus group, e.g. Human Immunodeficiency Virus types 1 and 2 (HIV-1 and HIV-2).

Retroviruses are RNA viruses which replicate through a DNA proviral intermediate which is integrated in the genome of the infected host cell. The virion particle contains a dimer of positive-strand genomic RNA molecules. This genomic RNA is the full-length species transcribed from the proviral DNA by the host RNA polymerase II. A proportion of these full-length RNAs which encode the gag and pol genes of the virus is translated by the host cell ribosomes, to produce the structural and enzymic proteins required for production of virion particles. The provirus also gives rise to a variety of smaller singly and multiply-spliced mRNAs coding for the envelope proteins and, in the case of more complex retroviruses, a group of regulatory proteins. The genomic (and subgenomic) RNA molecules are structurally similar to cellular mRNAs in having a 5′ m⁷G cap and a polyadenylated 3′ tail.

A series of problems must be addressed for successful packaging of genomic RNA. The full-length RNA must be packaged preferentially over the spliced viral messages as it is the only one carrying the full complement of genetic information for the next generation of virions. The virus mast also specifically select the genomic RNA against the enormous quantity and variety of physically similar host cell mRNAs as, unlike many other viruses, retroviruses do not generally arrest host RNA synthesis. Genomic RNA constitutes approximately 1% of the total RNA in an infected cell yet is the major species incorporated into virus particles. There must be a mechanism whereby genomic RNA to be packaged is recognised such that a proportion is either protected from being translated and transported to an assembly site or is associated with the gag precursor polyprotein which it has encoded immediately after translation. Lastly, there is the stoichiometric problem of having to package the correct number of genomes in association with 3-4000 gag precursor proteins, adequate numbers of reverse transcriptase molecules, a protease, tRNA primers and, in some cases, multiple copies of regulatory proteins.

Packaging the genome thus entails problems of specificity of selection of RNA and also considerations of RNA compartmentalisation.

The virus overcomes these problems by the presence of cis-acting elements, i.e. “packaging signals”, in the viral genomic mRNA and by protein factors acting in trans. Studies on spontaneously arising and laboratory constructed viral mutants have confirmed that specific sequences are critical for RNA recognition and packaging. Linial et al, Cell 15:1371-1381 (1978); Mann et al, Cell 33:153-159 (1983); Watanabe et al, PNAS USA 79:5986-5990 (1979) and WO-A-9119798 disclose that deletions in the 5′ untranslated leader sequence lead to defects in packaging in, respectively, Rous Sarcoma Virus (RSV), Moloney Murine Leukemia Virus (MoMLV), Spleen Necrosis Virus (SNV) and HIV.

In the case of HIV-1, the viral Gag polyprotein in its uncleaved state specifically recognises and binds to RNAs that contain the Ψ packaging signal (Kaye and Lever, J. Virology 70:880-886 (1996)). HIV-1 appears to be able to perform this function without being translated in cis from the viral genome (McBride et al., J. Virology 71:4544-4554 (1997)), allowing HIV-1 to be successfully used as a gene vector system.

There is a non-reciprocal relationship in the ability of HIV-1 and HIV-2 to package each others RNA: wild-type HIV-2 is only able to package its own RNA, whereas HIV-1 efficiently packages both HIV-1 and HIV-2 based vector constructs in addition to its own RNA genome (Kaye and Lever, J. Virology 72:5877-5885 (1998)). HIV-2 clearly only packages its own genomic RNA. The mechanism employed to achieve this is co-translational; that is only genomic HIV-2 RNAs which are templates for a full length Gag polyprotein containing an intact nucleocapsid region are efficiently incorporated into progeny virions (Kaye and Lever, J. Virology 73:3023-3031 (1999)). This mechanism explains the inability of wild-type HIV-2 to package either HIV-1 or HIV-2 based vectors in trans, and would seemingly preclude HIV-2 from use as a gene vector.

Deletion and substitution mutagenesis have defined sequences necessary for RNA packaging in several retroviruses. In some of these, the extent of the sequence sufficient for packaging has also been mapped. Implicit in the description of packaging signals and RNA secondary structure is the premise that, if this sequence is introduced into heterologous RNA then, theoretically, the heterologous RNA should be packaged by retroviral particles. Constraints on packaging include the theoretical one (for which Mann et al, J. Virol. 54:401-407 (1985), provide some circumstantial evidence) that sequences adjacent to the packaging signal (PSI) should not favour the formation of alternative secondary structures disrupting PSI. Additionally, the total length of RNA packaged is physically limited by the capacity of the virus to package RNA of a certain size. In HIV-1, proviral constructs incorporating heterologous genes have been shown by Terwilliger et al. PNAS USA 86:3857-3861 (1989), to lead to a replication defect when the total length of the viral RNA produced significantly exceeds that of the original virus. The replication defect is consistent with a declining efficiency of RNA packaging.

Nevertheless, there is significant variability between different viruses in the nature and site of their packaging sequences. The mechanism of RNA recognition is so poorly understood that theoretically it is not possible to make predictions of the exact site and nature of packaging sequences without experimental data.

The development of retroviral vector systems has been a direct development of the work described above. In these systems, a packaging-defective “helper” virus is used to generate particles which package a highly modified RNA genome (the vector). Watanabe et al, Mol. Cell Biol. 3:2241-2249 (1983), and Eglitis et al, BioTechniques 6:608-614 (1988), report that vectors containing a minimum of the viral long terminal repeats, the packaging signal and a primer-binding site together with a heterologous marker gene have been packaged into virion particles and transferred to the cells for which the parent virus is tropic. By this means, it has been possible to define the minimal sequence required for packaging of RNA into a virus particle.

### SUMMARY OF THE INVENTION

In one aspect, the present invention provides a Human Immunodeficiency Virus (HIV-2) vector comprising a mutation within an HIV-2 packaging signal such that viral RNA is not packaged within an HIV-2 capsid. The vector has a mutation comprising deletion of
(a) a sequence of SEQ ID NO:1 or a variant thereof,
(b) an internal fragment thereof of 5 or more nucleotides in length, or
(c) a fragment thereof of 17 or more nucleotides in length.

In another aspect of the present invention, there is provided a vector comprising an 17-2 packaging signal and a heterologous gene capable of being expressed in the vector.

The invention also provides a process for producing an HIV-2 virus encoding a heterologous gene, which process comprises infecting a host cell with a vector which is capable of producing an HIV-2 capsid and a vector according to the invention capable of expressing a heterologous gene and having HIV-2 packaging sequences sufficient to package the vector in the HIV-2 capsid; and culturing the host cell. Viruses produced in accordance with the invention can be used to deliver the heterologous gene to a host cell, for example in a method of gene therapy, vaccination or in scientific investigation.

In another aspect of the present invention, an HIV-2 packaging sequence is used in the treatment or prophylaxis of SIV or HIV infection.

### DESCRIPTION OF THE FIGURES

FIG. 1—Predicted RNA secondary structure of HIV-2-leader RNA

FIG. 2—Schematic of the locations of DM and Ψ1 deletions in the HIV-2 leader, as well as those of the pSVRΔ1, Δ2, Δ3 and Δ4 deletions.

FIG. 3—Reduction of mutant packaging efficiencies in competition with wild-type HIV-2. Bar chart shows quantification of packaging efficiencies of mutants with and without competition. Results are averages of at least three separate experiments, error bars show the standard error of the mean between experiments.

FIG. 4—Packaging of wild-type HIV-2 assessed by RPA when co-transfected with a DM mutant virus that could produce Gag protein, compared to one that could not. Bar chart shows quantification of experiments. Packaging efficiency of pSVR in competition with a non-Gag producing virus, pSVRDMΔH is taken as 100%. Results are the average of four separate experiments, error bars represent the standard error of the mean between experiments.

FIG. 5—Structures of two HIV-2 based helper virus constructs and two HIV-2 vectors containing a puromycin resistance gene cassette under the control of the Simian Virus 40 promoter.

FIG. 6—Ability of pSVRDM to trans-package HIV-2 vectors containing various amounts of the gag ORF. Quantification of vector packaging efficiencies. Results are average of at least two separate experiments, error bars represent the standard error of the mean between experiments.

FIG. 7—Vector packaging efficiencies following competition for limiting amounts of Gag polyprotein. Results are the average of three separate experiments, error bars represent the standard error of the mean between experiments.

### DESCRIPTION OF THE INVENTION

Packaging Defective HIV-2 Vectors

The present invention is based on studies which have identified packaging signals in the HIV-2 genome. Such packing sequences can be deleted to produce an HIV-2 vector which cannot itself be packaged into the HIV-2 capsid. Such HIV-2 vectors can be used to produce HIV-2 capsids. Host cells co-transformed with vectors which incorporate the HIV-2 packaging signals can then be used to generate capsid into which such vectors are packaged to produce an HIV capsid having nucleotide sequences therein capable of expressing heterologous proteins. The present invention provides a vector which is packaging defective. Such vectors may also comprise other mutations within other HIV-2 genes. Preferably such vectors retain the ability to express and assemble the HIV-2 capsid.

A packaging-defective deficient vector should contain an HIV-2 nucleotide segment containing a sufficient number of nucleotides corresponding to nucleotides of the HIV-2 genome to express functional HIV-2 gene products, but as described above, should not contain a sufficient number of HIV-2 nucleotides to permit efficient packaging of the viral RNA into virions.

HIV-2 has been described in a number of references. For example, McCann and Lever, J Virology 71: 4133-4137 (1997) disclose pSVR which is in an infectious proviral clone of the ROD strain of HIV-2 containing the replication origin of simian virus 40. HIV-2 nucleotide positions herein are numbered relative to the first nucleotide of the viral RNA, that is, the transcript start site is defined as 1.

SEQ ID NO: 1 comprises positions 380-408 of the HIV-2 RNA and has been demonstrated as being important for packaging of HIV-2 in accordance with the present invention. The 28 based nucleotide sequence of SEQ ID NO: 1 is AACAAACCACGACGGAGTGCTCCTAGAA.

Preferably, a packaging-defective vector of the invention comprises an HIV-2 genome which has been modified to comprise at least a deletion or mutation of (a) a sequence of SEQ ID NO: 1 or a fragment thereof, (b) an internal fragment thereof of 5 or more nucleotides in length, or (c) a fragment thereof of 17 or more nucleotides in length.

A mutation may comprise a deletion or modification of the sequence of SEQ ID NO: 1. An appropriate modification may comprise a substitution, addition and/or deletion. An appropriate mutation will be one which leads to a reduction in the ability of viral RNA to be packaged within an HIV-2 capsid. Preferably, such a mutation will lead to viral RNA not being packaged within an HIV-2 capsid.

The mutation may alternatively comprise deletion or modification of a fragment of SEQ ID NO: 1 or a variant thereof of 5 or more nucleotides in length. Such a fragment is an internal fragment, that is to say, a deletion of 5 or more nucleotides within SEQ ID NO: 1, not including the end nucleotides of SEQ ID NO: 1. Such a fragment may be, for example, 5, 10, 15, 20 or 25 nucleotides in length. In the alternative, the fragment may comprise a fragment of 17 or more nucleotides in length, selected from any portion of SEQ ID NO: 1 or a variant thereof including a terminal fragment thereof. Such a fragment may be, for example, 17, 19, 21, 23, 25, or 27 nucleotides in length.

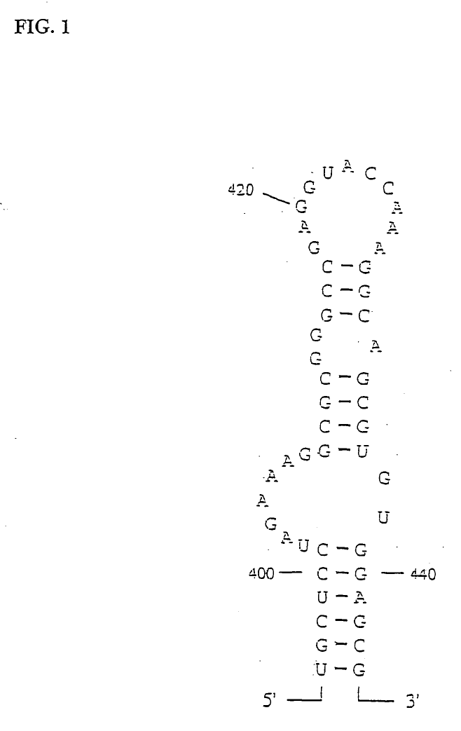

Alternatively larger deletions may be incorporated. Preferably, a larger deletion will comprise positions 380-408 of the HIV-2 RNA and will extend from this location in one or both directions. Such a deletion may comprise a deletion of, for example, 1, 2, 5, 10, 20, 30, 50 or more bases at one or both ends of this sequence. This region of the HIV-2 genome includes a proposed structural fold as shown in FIG. 1, and is associated with a palindromic terminus. Preferably the deletion will disrupt the formation of the palindromic terminus and thus remove this structure. Preferably a deletion will lie between the primer binding site and this proposed structural fold.

A variant of the sequence identified in SEQ ID NO; 1 is a corresponding sequence derived from a variant HIV-2 genome which may be identified, for example, by identifying the major 5′splice donor site, primer binding site or gag initiation codon of a variant HIV-2 genome and aligning the sequence of the variant to SEQ ID NO: 1 or to the sequence of the HIV-2 genome described in McCann and Lever (supra) to identify the corresponding sequence of the variant HIV-2 genome to SEQ ID NO: 1.

The HIV-2 genome as used herein refers to the viral RNA derived from an HIV-2. The human immunodeficiency viruses (HIV-2) of the invention may be derived from any HIV-2, strain or derivatives thereof. Derivatives preferably have at least 70% sequence homology to the HIV-2 genome, more preferably at least 80%, even more preferably at least 90 or 95%. Other derivatives which may be used to obtain the viruses of the present invention include strains that already have mutations in some HIV-2 genes. Other mutations may also be present as set out in more detail below. The position of locations such as the primer binding site and 5′ major splice donor site can readily be established by one skilled in the art by reference to the published HIV-2 sequences or for example by aligning a variant HIV-2 to the sequences set out and described herein.

In accordance with one aspect of the invention an HIV-2 vector comprises a mutation within an HIV-2 packaging signal such that the mutated HIV-2 RNA is not packaged within the HIV-2 envelope protein or capsid. In particular, it is preferred that a packaging defective vector of the present invention comprises sufficient means to express functional HIV-2 envelope proteins and to produce HIV-2 capsids. Further deletions in the HIV-2 genome may be incorporated into the vector such as deletions of polymerase so that replication of the HIV-2 genome cannot occur should it be packaged into the capsid.

Vectors Comprising HIV-2 Packaging Sequences

It has now been shown that Gag protein produced from packaging-defective vectors will find another RNA which comprises a packaging signal to package. Thus, vectors which include HIV-2 packaging sequences but are unable to either package themselves or be packaged by wild-type HIV-2 are able to compete for Gag made by HIV-2 vectors which lack packaging signals. Packaging in HIV-2 is particularly tightly controlled. Gag protein will selectively package RNA with packaging signals in preference to any other RNA. Levels of Gag protein are limiting so only RNA with the highest affinity signals will be packaged. This is in marked contrast to HIV-2 in which Gag protein is in vast excess and the virus particles produced may contain any RNA with high or low affinity packaging signals. Because of the tight control on packaging in HIV-2, virus preparations will be of high purity and less likely to contain unwanted nucleic acids. The vectors of the present invention are therefore particularly useful for the delivery of heterologous genes or the production of capsids containing heterologous genes. They are therefore ideally suited for use in somatic gene therapy.

The vectors comprising HIV-2 packaging sequences may be capable of being packaged by the HIV-2 envelope or heterologous viral envelopes such as the Amphotrophic Murine Leukaemia Virus envelope of the Vesicular Stomatitis Virus G protein (VSV-G). These vectors may be capable of being packaged by HIV-1.

The invention additionally relates to a vector for expression of a heterologous gene which may be packaged into the HIV-2 genome through the use of HIV-2 packaging sequences. Such a vector may comprise any suitable vector compatible with the proposed administration or use of the virus so long as HIV-2 packaging sequences are incorporated. Preferably the vector is derived from the HIV-2 genome but includes mutation in one or more HIV-2 genes, for example, to render the HIV-2 genome replication deficient.

Preferably the packaging sequences present in such a vector correspond to those described above which are mutated to produce a packaging defective HIV-2 vector. Preferably a substantial portion of the packaging signal is included. In a preferred aspect, the packaging sequence comprises the sequence of SEQ ID NO: 1, or a fragment thereof or a variant thereof. A variant thereof may be identified as set out above in determining a region of the genome to be deleted. All of the sequences described above for mutation or deletion to produce an HIV-2 packaging defective vector are preferred sequences for incorporation into a vector such that the vector can be packaged by an HIV-2 capsid or protein envelope. In a preferred aspect, the packaging sequence is selected to allow the formation of a palindromic terminus, having the structure as shown in FIG. 1.

In addition to the packaging sequences described above, further HIV-2 packaging sequences may be present in a vector. These sequences may comprise 10, 20, 50, 100, 200, 300 or 400 or more polynucleotides from a region downstream of the 5′ splice donor site. In a preferred aspect, these packaging sequences comprise the 5′ part of gag, preferably comprising the matrix (MA) region of the gag ORF. In a preferred aspect, the packaging sequence comprises the sequence that lies between positions 553 and 912 of the HIV-2 RNA, or a variant thereof. A variant of such a packaging sequence is a corresponding sequence derived from a variant HIV-2 genome which may be identified, for example, by identifying the major 5′splice donor site, primer binding site or gag initiation codon of a variant HIV-2 genome and aligning the sequence of the variant to the sequence of the HIV-2 genome described in McCann and Lever (supra) to identify the corresponding sequence of the variant HIV-2 genome to SEQ ID NO: 2.

These vectors may be used as an extremely efficient way to package desired genetic sequences and deliver them to target cells infectable by HIV-2. This may be done by preparing a vector containing a nucleotide segment containing a sufficient number of nucleotides corresponding to the packaging nucleotides of HIV-2 (HIV-2 packaging region), a predetermined gene and, flanking the packaging sequence and predetermined gene, sequences corresponding to a sufficient number of sequences from within and near the LTR for packaging, reverse transcription, integration of the vector into target cells and gene expression from the vector.

The packaging region preferably corresponds to at least the sequence of SEQ ID NO: 1. With regard to the experimental data presented below concerning the packaging of such a vector, the vector might also comprise the 5′ part of gag, preferably including the matrix (MA) sequence of HIV-2 in order to enhance packaging efficiency. For example, a sufficient number of HIV-2 sequences to be packaged, reverse-transcribed, integrated into and expressed in the target cells would include the U3, R and U5 sequences of the LTRs, the packaging sequences and some sequences flanking the LTRs (required for reverse transcription). Mutation of the gag initiation codon might be acceptable to avoid translation starting from this point whilst still retaining the cis acting gag nucleotide sequence required for packaging. For example, the gag ATG may be changed to ATC by site-directed mutagenesis.

When this vector is used to transfect an HIV-2 packaging-deficient cell, it is the nucleotide sequence from this vector that will be packaged in the virions produced. These HIV-2 packaged genes may then be targeted to cells infectable by HIV-2. This method of transformation is expected to be much more efficient than current methods. Further, by appropriate choice of genes, the method of HIV-2 infection may be monitored.

For example, the vector could contain a sufficient number of nucleotides corresponding to both 5′ and 3′ LTRs of HIV-2 to be expressed, reverse-transcribed and integrated, a sufficient number of nucleotides corresponding to the HIV-2 packaging sequences to be packaged. The vector would also contain a sufficient number of nucleotides of the gene which is desired to be transferred to produce a functional gene (e.g. gene segment). This gene can be any gene desired, as described below. The vector may also contain sequences corresponding to a promoter region which regulates the expression of the gene. The vector may be a self-inactivating vector, for example a self-inactivating retroviral vector. This may comprise a mutation in the U3 region of the 3′LTR of the vector which, after infection of the target cell during reverse transcription, is copied so that the 5′ LTR contains this inactivating mutation, and the lone terminal repeat promoter is inactivated. This leaves any internal promoter to function independently of any competition.

Packaging Sequences

In an alternative aspect of the present invention HIV-2 packaging sequences may be provided on their own or as anitisense molecules to interfere with packaging of wild type HIV-2 or to interfere with packaging of HIV-2 in an HIV-2 capsid. Thus the packaging sequences may be used in the prophylaxis or treatment of SIV or infection, such as SIV, HIV-1 or HIV-2 infection and preferably HIV-1 infection. In particular packaging sequences such as those described either for deletion above or for incorporation with a vector for expression of heterologous genes below may be used either alone or for the generation of antisense molecules as described in more detail below. The packaging sequences may be provided in a suitable delivery vehicle for example flanked by non-SIV or HIV sequences. Such packaging sequences can be used to bind to SIV or HIV capsid proteins or to saturate such binding sites or compete for such sites with wild type viral genome and thus prevent packaging of such genomes in the capsid. Thus the packaging sequences may be useful in therapy in their own right. Antisense molecules can be used to bind to wild type viral genome packaging sequences and thus prevent their recognition and binding with the viral capsid. Such packaging nucleotides may be formulated as described below or may be administered as naked polynucleotides or formulated with transfection facilitating agents as is well known in the art and delivered by any suitable technique.

Preferably the packaging sequences correspond to those described above which are mutated to produce a packaging defective HIV-2 vector. A substantial portion of the packaging signal may be included. In a preferred aspect the packaging sequence comprises the sequence of SEQ ID NO: 1 or a fragment thereof or a variant thereof. In particular the packaging sequence is selected to allow the formation of a palindromic terminus, having the structure as shown in FIG. 1. A variant thereof may be identified as set out above in determining a region of the genome to be deleted. All of the sequences described above for mutation or deletion to produce a HIV-2 packaging defective vector are preferred sequences for incorporation into a vector such that the vector can be packaged by an HIV-2 capsid or protein envelope. Additional sequences are also preferably provided such as 10, 20, 50, 100, 200, 300 or 400 or more polynucleotides from a region downstream of the 5′ splice donor site. In a preferred aspect, the packaging sequences comprise the 5 part of gag, preferably comprising the matrix (MA) region of the gag ORF. In a preferred aspect, the packaging sequence comprises the sequence that lies between positions 553 and 912 of the HIV-2 RNA.

Alternatively or additionally, other flanking sequences may be provided for delivery of the packaging sequences. Antisense molecules which are complementary to the packaging sequences described herein may also be provided.

Host Cells

In one aspect of the present invention, host cells are generated to produce HIV-2 virus containing a vector for expression of a heterologous gene. The viruses are produced by co-transfecting a cell with a vector which is capable of producing an HIV-2 capsid and a vector according to the invention having an HIV-2 packaging signal and a heterologous gene. In a preferred aspect, the vector which is capable of producing an HIV-2 capsid is a packaging defective HIV-2 vector according to the invention. Such viruses are produced by co-transfecting a suitable cell such as a mammalian cell with both vectors.

Preferably, a selected cell line is transformed using at least two different vectors, each containing a different portion of the HIV-2 genome and also not containing the sequence necessary for viral packaging. Then, by co-transfecting a cell with each vector, the cell would still be able to express all the HIV-2 structural and enzymatic proteins and produce virions. In one preferred embodiment the, or each, vector does not contain sequences corresponding to an HIV-2 LTR (long terminal repeat sequence) but contains sequences corresponding to a promoter region and/or another genome's polyadenylation sequences. The, or each, vector may be a self-inactivating vector. This may, for example, comprise a mutation in the U3 region of the 3′LTR of the vector which, after infection of the target cell during reverse transcription, is copied so that the 5′ LTR contains this inactivating mutation and the long terminal repeat promoter is inactivated. This leaves any internal promoter to function independently of any competition. Selection of particular promoters and polyadenylation sequences can readily be determined based upon the particular host cell. Preferably, the LTR to which the sequences do not correspond is the 3′LTR.

In one preferred embodiment, one vector includes sequences permitting expression of HIV-2 proteins upstream of env and the second vector permits expression of the remaining proteins. For example, one vector contains an HIV-2 nucleotide segment corresponding to a sufficient number of nucleotides upstream of the gag initiation codon to the env gene sequence to express the 5′-most gene products. The other vector contains an HIV-2 nucleotide segment corresponding to a sufficient number of nucleotides downstream of the gag gene sequence and including a functional env gene sequence. Such vectors can be chemically synthesised from the reported gene sequence of the HIV-2 genome or derived from the many available HIV-2 proviruses, by taking advantage of the known restriction endonuclease sites in these viruses by the skilled artisan based on the present disclosure.

Preferably, a different marker gene is added to each vector. Then, using a preselected cell line cotransfected with these different vectors, and by looking for a cell containing both markers, a cell that has been cotransfected with both vectors is found. Such a cell would be able to produce all of the HIV-2 proteins. Although virions would be produced, the RNA corresponding to the entire viral sequences would not be packaged in these virions. One can use more than two vectors, if desired, e.g. a gag/pol vector, a protease vector and an env vector.

Retroviruses can in some cases be pseudotyped with the envelope glycoproteins of other viruses. Consequently, one can prepare a vector containing a sufficient number of nucleotides to correspond to an env gene from a different retrovirus. Preferably, the 5′LTR of this vector would be of the same genome as the env gene. Such a vector could be used instead of an HIV-2 env packaging-defective vector, to create virions. By such a change, the resultant vector systems could be used in a wider host range or could be restricted to a smaller host range. Using a vesicular stomatitis virus or rabies virus envelope protein would make the vector tropic for many different cell types.

Virtually any cell line can be used. Preferably, a mammalian cell line is used, for example CV-1, Hela, Raji, SW480 or CHO.

In order to increase production of the viral cellular products, one could use a promoter other than the 5′ LTR, e.g. b) replacing the 5′ LTR with a promoter that will preferentially express genes in CV-1 or HeLa cells. The particular promoter used can easily be determined by the person of ordinary skill in the art depending on the cell line used, based on the present disclosure.

In order to enhance the level of iral cellular products, one can also add enhancer sequences to the vector to get enhancement of the HIV-2 LTR and/or promoter. Particular enhancer sequences can readily be determined by a person of ordinary skill in the art depending on the host cell line.

By using a series of vectors that together contain the complete HIV-2 genome, one can create cell lines that produce a virion that is identical to the HIV-2 virion except that the virion does not contain HIV-2 RNA. These virions can readily be obtained from the cells. For example, the cells are cultured and the supernatant harvested. Depending on the desired use, the supernatant containing the virions can be used or these virions can be separated from the supernatant by standard techniques such as gradient centrifugation, filtering etc.

These attenuated virions are extremely useful in preparing a vaccine. The virions can be used to generate an antibody response to HIV-2 virions and, because these virions are identical to the actual HIV-2 virions except that the interior of these virions do not contain the viral RNA, the vaccine created should be particularly useful. Pseudotyped virions produced from cell lines cotransfected with HIV-2 gag/pol and protease genes and containing the env gene from another virus may be useful in creating a vaccine against this other virus. For example, an SIV env vector in the cell may give rise to a viral particle with an SIV env capable of eliciting an antibody response to SIV but without pathogenicity because of the absence of any other SIV proteins or SIV RNA.

Methods of Mutation

Mutations may be made in HIV-2 by homologous recombination methods well known to those skilled in the art. For example, HIV-2 genomic RNA is transfected together with a vector, preferably a plasmid vector, comprising the mutated sequence flanked by homologous HIV-2 sequences. The mutated sequence may comprise deletions, insertions or substitutions, all of which may be constructed by routine techniques. Insertions may include selectable marker genes, for example lacZ, for screening recombinant viruses by, for example, β-galactosidase activity.

The number of bases that need to be deleted or mutated can vary greatly. For example, the given 28-base pair deletion in HIV-2 is sufficient to result in loss of packaging ability. However, even smaller deletions in this region could also result in loss of packaging efficiency. Indeed, it is expected that a deletion as small as about 5, 10, 15, 17, 18, 19, 20, 25, 26 or 27 bases in this region can remove efficient packaging ability. The mutation may comprise deletion or modification of a fragment of SEQ ID NO: 1 or a variant thereof of 5 or more nucleotides in length. Such a fragment is an internal fragment, that is to say, a deletion of 5 or more nucleotides within SEQ ID NO: 1, not including the end nucleotides of SEQ ID NO: 1. In the alternative, the mutation may comprise deletion or modification of a fragment comprising 17 or more nucleotides in length, selected from any portion of SEQ ID NO: 1 or a variant thereof including a terminal fragment thereof. Alternatively larger deletions may be incorporated as described above. The size of a particular deletion can readily be determined based on the present disclosure by the person of ordinary skill in the art.

Essential genes may be rendered functionally inactive by several techniques well known in the art. For example, they may be rendered functionally inactive by deletions, substitutions or insertions, preferably by deletion. Deletions may remove portions of the genes or the entire gene. For example, deletion of only one nucleotide may be made, resulting in a frame shift. However, preferably larger deletions are made, for example at least 25%, more preferably at least 50% of the total coding and non-coding sequence (or alternatively, in absolute terms, at least 10 nucleotides, more preferably at least 100 nucleotides, most preferably, at least 1000 nucleotides). It is particularly preferred to remove the entire gene and some of the flanking sequences. Inserted sequences may include the heterologous genes described below.

Heterologous Genes and Promoters

A vector or viruses of the invention may be modified to carne a heterologous gene, that is to say a gene other than one present in the HIV-2 genome. In particular the invention provides vectors which have HIV-2 derived sequences sufficient to allow packaging of the vector into a HIV-2 capsid. The vectors may be derived from HIV-2 genomes, incorporating mutations or deletions in one or more HIV-2 genes, or may be derived from other expression vectors which are modified to incorporate HIV-2 packaging sequences. The term “heterologous gene” comprises any gene other than one present in the HIV-2 genome. The heterologous gene may be any allelic variant of a wild-type gene, or it may be a mutant gene. The term “gene” is intended to cover nucleic acid sequences which are capable of being at least transcribed. Thus, sequences encoding mRNA, tRNA and rRNA are included within this definition. The sequences may be in the sense or antisense orientation with respect to the promoter. Antisense constructs can be used to inhibit the expression of a gene in a cell according to well-known techniques. Sequences encoding mRNA will optionally include some or all of 5′ and/or 3′ transcribed but untranslated flanking sequences naturally, or otherwise, associated with the translated coding sequence. It may optionally further include the associated transcriptional control sequences normally associated with the transcribed sequences, for example transcriptional stop signals, polyadenylation sites and downstream enhancer elements.

The heterologous gene may be inserted into for example an HIV-2 vector by homologous recombination of HIV-2 strains with, for example, plasmid vectors carrying the heterologous gene flanked by HIV-2 sequences. The heterologous gene may be introduced into a suitable plasmid vector comprising HIV-2 sequences using cloning techniques well-known in the art. The heterologous gene may be inserted into an HIV-2 vector at any location. It is preferred that the heterologous gene is inserted into an essential HIV-2 gene. Preferably the vector is derived from an HIV-2 genome, but includes deletion of one, two or several of the HIV-2 genes, up to the minimal sequences of the HIV-2 genome to provide for packaging and expression of the heterologous gene.

The transcribed sequence of the heterologous gene is preferably operably linked to a control sequence permitting expression of the heterologous gene in mammalian cells. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence.

The control sequence comprises a promoter allowing expression of the heterologous gene and a signal for termination of transcription. The promoter is selected from promoters which are functional in mammalian, preferably human, cells. The promoter may be derived from promoter sequences of eukaryotic genes. For example, it may be a promoter derived from the genome of a cell in which expression of the heterologous gene is to occur. With respect to eukaryotic promoters, they may be promoters that function in a ubiquitous manner (such as promoters of β-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter or promoters of HIV-2 genes.

The HIV-2 LTR promoter, and promoters containing elements of the LTR promoter region, are especially preferred. The expression cassette may further comprise a second promoter and a second heterologous gene operably linked in that order and in the opposite or same orientation to the first promoter and first heterologous gene wherein said second promoter and second heterologous gene are the same as or different to the first promoter and first heterologous gene. Thus a pair of promoter/heterologous gene constructs may allow the expression of pairs of heterologous genes, which may be the same or different, driven by the same or different promoters. Furthermore, the product of the first heterologous gene may regulate the expression of the second heterologous gene (or vice-versa) under suitable physiological conditions.

The expression cassette can be constructed using routine cloning techniques known to persons skilled in the art (see, for example. Sambrook et al., 1989, Molecular Cloning—a laboratory manual; Cold Spring Harbor Press).

It may also be advantageous for the promoters to be inducible so that the levels of expression of the heterologous gene can be regulated during the life-time of the cell. Inducible means that the levels of expression obtained using the promoter can be regulated. For example, in a preferred embodiment where more than one heterologous gene is inserted into the vector or HIV-2 genome, one promoter would comprise a promoter responsive to the expression of the second protein and driving the heterologous gene the expression of which is to be regulated. The second promoter would comprise a strong promoter (e.g. the CMV IE promoter) driving the expression of the second protein.

In addition, any of these promoters may be modified by the addition of further regulatory sequences, for example enhancer sequences. Chimeric promoters may also be used comprising sequence elements from two or more different promoters described above, for example an MMLV LTR/HIV-2 fusion promoter.

The heterologous gene may encode, for example, proteins involved in the regulation of cell division, for example mitogenic growth factors, cytokines (such as α-, β- or γ-interferon, interleukins including IL-1, IL-2, tumour necrosis factor, or insulin-like growth factors I or II), protein kinases (such as MAP kinase), protein phosphatases and cellular receptors for any of the above. The heterologous gene may also encode enzymes involved in cellular metabolic pathways, for example enzymes involved in amino acid biosynthesis or degradation (such as tyrosine hydroxylase), or protein involved in the regulation of such pathways, for example protein kinases and phosphatases. The heterologous gene may also encode transcription factors or proteins involved in their regulation, membrane proteins (such as rhodopsin), structural proteins (such as dystrophin) or heat shock proteins such as hsp27, hsp65, hsp70 and hsp90.

Preferably, the heterologous gene encodes a polypeptide of therapeutic use, or whose function or lack of function may be important in a disease process. For example, tyrosine hydroxylase can be used in the treatment of Parkinson's disease, rhodopsin can be used in the treatment of eye disorders, dystrophin may be used to treat muscular dystrophy, and heat shock proteins can be used to treat disorders of the heart and brain associated with ischaemic stress. Polypeptides of therapeutic use may also include cytotoxic polypeptides such as ricin, or enzymes capable of converting a precursor prodrug into a cytotoxic compound for use in, for example, methods of virus-directed enzyme prodrug therapy or gene-directed enzyme prodrug therapy. In the latter case, it may be desirable to ensure that the enzyme has a suitable signal sequence for directing it to the cell surface, preferably a signal sequence that allows the enzyme to be exposed on the exterior of the cell surface whilst remaining anchored to cell membrane.

Heterologous genes may also encode antigenic polypeptides for use as vaccines. Preferably such antigenic polypeptides are derived from pathogenic organisms, for example bacteria or viruses, or from tumours.

Heterologous genes may also include marker genes (for example encoding β-galactosidase or green fluorescent protein) or genes whose products regulate the expression of other genes (for example, transcriptional regulatory factors.

Gene therapy and other therapeutic applications may well require the administration of multiple genes. The expression of multiple genes may be advantageous for the treatment of a variety of conditions.

Administration

The vectors, host cells and viruses of the present invention may thus be used to deliver therapeutic genes to a human or animal in need of treatment.

One method for administered gene therapy involves inserting the therapeutic gene into a vector of the invention, as described above. Subsequently, cells are co-transfected in vitro with a vector comprising the heterologous gene and the HIV-2 packaging sequences and a packaging defective HIV-2 vector. Culturing the cells leads to production of HIV-2 viral capsids, into which the heterologous gene vectors are packaged through the HIV-2 packaging sequences. Because of the specific packaging competition shown here to occur in such an HILA-2 system, it is possible to eliminate the packaging of unwanted helper virus sequences in a much more rigorous way than is possible with other retroviral systems, for example the HIV-1 system. The resultant recombinant virus may be combined with a pharmaceutically acceptable carrier or diluent to produce a pharmaceutical composition. Suitable carriers and diluents include isotonic saline solutions, for example phosphate-buffered saline. Vaccine compositions, in which the heterologous gene encodes an antigenic peptide or protein may be formulated with adjuvants to enhance the immune response generated. The composition may be formulated for parenteral, intramuscular, intravenous, subcutaneous, intraocular or transdermal administration.

The pharmaceutical composition is administered in such a way that the virus containing the therapeutic gene for gene therapy, can be incorporated into cells at an appropriate area. The HIV-2 capsids containing the heterologous gene constructs are particularly useful due to the ability of HIV-2 to infect non dividing cells of many different types.

The amount of virus administered is in the range of from 10⁴ to 10¹⁰ pfu, preferably from 10⁵ to 10⁸ pfu, more preferably about 10⁶ to 10⁷ pfu. When injected, typically 1 to 10 μl of virus in a pharmaceutically acceptable suitable carrier or diluent is administered.

The routes of administration and dosages described are intended only as a guide since a skilled practitioner will be able to determine readily the optimum route of administration and dosage for any particular patient and condition.

Assay Methodologies

The viruses of the invention can also be used in methods of scientific research. Thus, a further aspect of the present invention relates to methods of assaying gene function in mammalian cells, either in vitro or in vivo. The function of a heterologous gene could be determined by a method comprising:
(a) producing virus particles comprising an HIV-2 capsid and vector having a heterologous gene packaged via HIV-2 packaging signals, and
(b) introducing the resulting virus into a mammalian cell line; and
(c) determining the effect of expression of said heterologous gene in said mammalian cell-line.

For example, the cell-line may have a temperature-sensitive defect in cell division. When an HIV-2 strain comprising a heterologous gene according to the invention is introduced into the defective cell-line and the cell-line grown at the restrictive temperature, a skilled person will easily be able to determine whether the heterologous gene can complement the defect in cell division. Similarly, other known techniques can be applied to determine if expression of the heterologous gene can correct an observable mutant phenotype in the mammalian cell-line.

This procedure can also be used to carry out systematic mutagenesis of a heterologous gene to ascertain which regions of the protein encoded by the gene are involved in restoring the mutant phenotype.

This method can also be used in animals, for example mice, carrying so-called “gene knock-outs”. A mild-type heterologous gene can be introduced into the animal using a mutant HIV-2 strain of the invention and the effect on the animal determined using various behavioural, histochemical or biochemical assays known in the art. Alternatively, a mutant heterologous gene can be introduced, into either a wild-type or “gene knock-out” animal to determine if disease-associated pathology is induced. An antisense nucleotide could also be introduced using the virus particle of the invention to create in effect a knock-out animal.

Alternatively, the mutant HIV-2 virus of the invention may be used to obtain expression of a gene under investigation in a target cell with subsequent incubation with a test substance to monitor the effect of the test substance on the target gene.

Thus, the methods of the present invention may be used in particular for the functional study of genes implicated in disease.

The invention will be described with reference to the following Examples, which are intended to be illustrative only and not limiting.

### EXAMPLE 1

Deletion of regions located upstream of the major splice donor have been shown to significantly reduced packaging efficiency in transient transfection of COS-1 cells (McCann and Lever, J. Virology 71:4133-4137 (1997); Kaye and Lever, J. Virology 73: 3023-3031 (1999)). There remains some controversy as to the exact location of the major packaging signal (Ψ) in HIV-2. This lack of consensus reflects the fact that the phenotypes of the various mutations have not been as profound as those reported in the HIV-1 system, or the deletions themselves have been too large to identify a discrete signal. Further deletions were therefore introduced in order to analyse regions that have not been characterised in previous investigations.

pSVR is an infectious proviral clone of the ROD strain of HIV-2 containing the replication origin of simian virus 40 (McCann and Lever (1997), as above). Restriction sites, where given are numbered relative to the first nucleotide of the viral RNA. Deletion mutations in the 5′ leader were introduced by site-directed mutagenesis following the Kunkel method (Kunkel et al, Methods Enzymol 154:367-382 (1987)) into a subclone of HIV-2, pGPAXS (Kaye and Lever, J. Virology 72:5877-5885 (1998)).

Proviral constructs pSVRΔ1, 2, 3 and 4 contain deletions in the 5′ leader region and are described in McCann and Lever, J. Virology 71: 4133-4137 (1997). For pSVRΔ1, positions 359-385 are deleted and for pSVRΔ2, positions 392-434 are deleted, both upstream of the major splice donor. For pSVRΔ3, positions 499-526 are deleted and for pSVRΔ4 positions 494-533 are deleted, both downstream of the major splice donor.

Further deletions were designed based on available structural information generated by computer modelling and biochemical analysis of the HIV-2 leader RNA (Berkhout and Schoneveld, Nucleic Acids Res 21:1117-1178 (1993); Damgaard et al., Nucleic Acids Res 26: 3667-3676 (1998)).

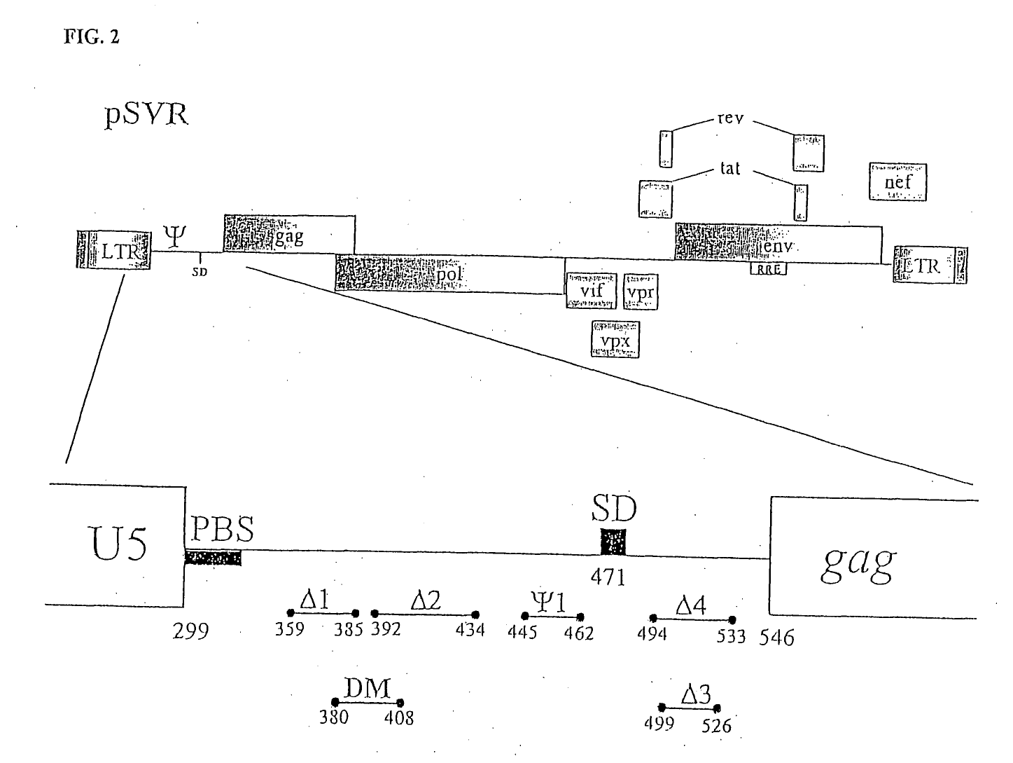

The first deletion, Ψ1, was designed to remove a predicted stem-loop from position 445-462. These positions were deleted using the mutagenic oligonucleotide 5′-GGCAGCGTGGAGCGGGGTGAAGGTAAGTACC-3′. The second, DM, 380-408 nt, overlaps with deletions pSVRΔ1 and pSVRΔ2 (FIG. 2). The DM deletion was made using the mutagenic oligonucleotide 5′ GGCAGTLAGGGCGGCAGGAGCGCGGGCCGAGGTACCAAAGGC-3′. The regions deleted in Ψ1 and DM are both located upstream of the major splice donor (position 472). Sequences from the resultant subclones pGRAXΨ1 and pGRADM containing the deletions were then introduced into the provirus by exchanging an Aat II (position 1384)-Xho I (position 2032) fragment. A double deletion mutant of both regions was also constructed, DM/Ψ1, by mutating pGRAXDM using the Ψ1 oligonucleotide and introducing this into the provirus.

Proviral clones containing these mutations were used to transiently transfect COS-1 cells. RNA from cytoplasmic and virion fractions was then analysed by RPA to assess any effects of the deletions on packaging. The Ψ1 mutation had only a very minor effect on packaging efficiency, whereas the DM deletion had a profound effect on the level of RNA incorporated into progeny virions (Table 1) considerably greater than the pSVRΔ2 deletion previously described as reducing packaging to around 20% of the level of wild type HIV-2. This is consistent with the region deleted by the DM mutation containing the core Ψ element of the virus. In addition, the double mutation has a similar phenotype, confirming that the DM deletion causes a profound defect and that the Ψ1 deletion causes no additional defect in packaging. There is also no apparent lack of RNA available for packaging in the mutants relative to the wild type.

TABLE 1
Relative packaging efficiencies^{a} of new HIV-2 packaging mutants

| Mutant | Relative P.E. (%) | +/− Standard error (%) |
|---|---|---|
| Ψ1 | 73 | 7.8 |
| DM | 5.7 | 1.6 |

^{a}calculated as ratio of virion RNA to cytoplasmic RNA relative to that of wild type.

To ensure that the effects observed above for deletions on packaging were not due to aberrant protein production, COS-1-cells transfected with wild type or mutant proviruses were metabolically labelled with 355 methionine, and viral proteins immunoprecipitated from cellular and virion fractions using pooled immune sera from HIV-2 infected individuals (MRC AIDS reagent project). By comparing the mutants with the wild type provirus, protein production had not been affected by these deletion mutations. In the cellular fraction, significant amounts of viral Gag and Env polyprotein precursors were apparent, which had been predominantly cleaved into mature proteins in the virions present in the supernatant. This indicates that there is no apparent defect in post-translational processing of viral proteins caused by these deletions. Packaging defects are, therefore, unlikely to be caused by reduced availability of Gag polyprotein for packaging, or any defect in particle release from the cell surface. These observations have also been confirmed by western blotting using a monoclonal antibody to HIV-2 capsid (CA) protein (Chemicon), and were further supported by studies showing there being no significant difference between wild type and mutant reverse transcriptase activities in culture supernatants.

Mutations studied in model systems may not necessarily have the same phenotypes as in vivo. The effects of the Ψ1 and DM deletions on viral replication in a more physiologically relevant cell type there therefore examined. Supernatants from transfected COS-1 cells were prepared as described below, and used to infect Jurkat T-cells in replicate assays. Virion particle production was measured through time by reverse transcriptase activity present in the supernatant. Cultures infected with wild type virus showed a gradual increase in particle production that peaked at 14 days post-infection (dpi). After this point particle production decreased, probably due to a decline of surviving susceptible cell populations, there being no fresh cells added to the assay. Cells infected with Ψ1 mutant virus displayed an intermediate replication phenotype, with no discernible peak at 14 dpi. Tis is consistent with the same mild packaging defect observed in COS-1 cells retarding virus spread due to the release of fewer infectious particles into the culture. Virus production from cultures infected with virus containing the DM deletion, both alone, and in the context of the double mutant, was severely reduced. There was a Gradual decline from an already low initial level of particle production at 4 dpi to levels of reverse transcriptase activity at 21 dpi barely measurable above background. This indicates that the virus was unable to spread efficiently beyond those cells that were infected by the original inoculum. In addition, early reverse transcriptase readings indicated that infection initiated successfully, so it is unlikely that the DM deletion interferes with early events in the virus life cycle. The DM deletion, therefore, causes a replication phenotype in permissive cells solely attributable to a defect in RNA packaging.

### EXAMPLE 2

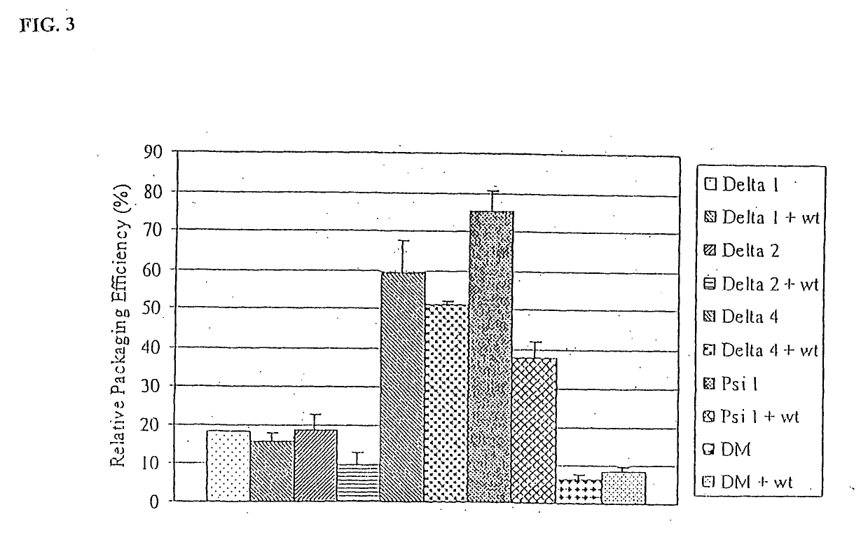

Co-transfection with wild type virus was used as an internal control for levels of RNA during packaging studies, as it enables mutants to be normalised to the wild type virus when calculating packaging efficiency (Kaye and Lever, Virology 73: 3023-3031 (1999); McBride and Panganiban, J. Virology 71:2050-2058 (1997)). Similar investigations were undertaken, in which equal amounts of wild type and mutant proviruses were co-transfected into COS-1 cells, and analysed the results by RPA. The packaging efficiencies of mutants possessing deletions located upstream of the splice donor were consistently reduced compared to when they were transfected alone. The largest reduction in packaging caused by competition was observed for the Ψ1 mutant, which was reduced from around 70% to 40% efficiency relative to wild type (FIG. 3). Both pSVRΔ1 and pSVRΔ2 deletion mutants were reduced, albeit less markedly, though these viruses are already quite severely deficient in packaging. A deletion located downstream of the splice donor, pSVRΔ4, also showed a slight decrease in packaging efficiency in competition, despite having only a mild effect on packaging itself. Packaging efficiency in the DM mutant is already so profoundly impaired even in the absence of competition, that detection of any chance is beyond the level of sensitivity of the assay. It appears, based on these observations, that Ψ region mutants are less efficient at targeting de novo synthesised Gag back to their own RNA in cis than is wild type HIV-2. Furthermore, wild type HIV-2 RNA with an intact Ψ region is able to compete for this Gag causing a reduction in mutant packaging efficiency.

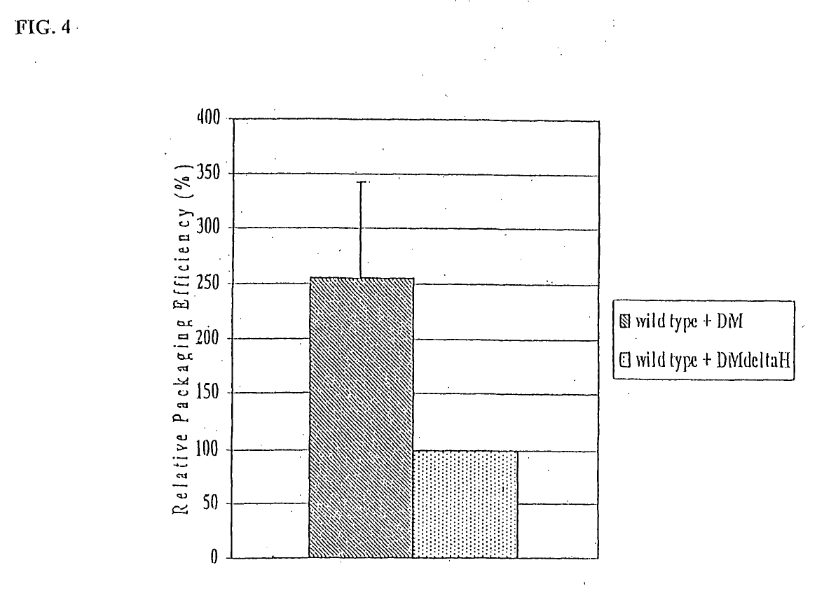

If competition effects reduce the packaging of Ψ region mutants, a logical question to ask is whether there is a corresponding increase in the packaging of wild type RNA. To address this, an HIV-2 provirus that contained the DM deletion and a premature stop codon in the Gag, ORF was constructed; pSVRDMΔH (as described below). It is known that HIV-2 vectors containing this stop mutation synthesise a truncated Gag polyprotein that is unable to incorporate RNA into virions (Kaye and Lever, 1999, as above). Such vectors are also unable to be efficiently packaged by wild type HIV-2 in trans. Co-transfection experiments were performed to compare the packaging of wild type HIV-2 RNA in competition with this virus or with pSVRDM, which is able to make its own full-length Gag. There should be twice the amount of Gag available in the cell in the latter. The results, as analysed ban RPA, are shown in (FIG. 4). As expected, wild rope HIV-2 in competition with a DM mutant that is able to make full-length Gag, was packaged around twice as efficiently, as when competing with a DM virus that cannot do so. As the levels of wild type RNA available for packaging in the cytoplasm will be the same in the two cases, it follows that the increase in efficiency observed is due to their being twice the amount of Gag present. Availability of Gag is, therefore, limiting for HIV-2 RNA packaging.

It is known that wild type HIV-2 is unable to efficiently package vectors in trans due to the use of a co-translational method of selecting its genomic RNA for packaging, termed cis-packaging. HIV-1, however, is able to do this efficiently and predominantly uses a trans-acting mechanism to select its genome for packaging, a strategy made possible due to the location of the core Ψ region in HIV-1 downstream of the major splice donor. Results of the competition experiments indicated that the. Gag being competed for was that made by the HIV-2 Ψ region mutants. This meant that such Gag was not being efficiently targeted in cis to its template RNA, and was therefore available to trans pathways. To test whether an HIV-2 Ψ region mutant can act as a helper virus and package an HIV-2 vector in trans, HIV-2 Ψ region mutants were co-transfected with a vector containing the stop mutation in Gag described above. All of the HIV-2 Ψ region mutants tested were able to efficiently incorporate vector RNA into virions, in contrast to the analogous experiments in which wild type HIV-2 was used as a helper virus. In addition, mutants with deletions downstream of the splice donor were also able to efficiently package vector RNA in trans.

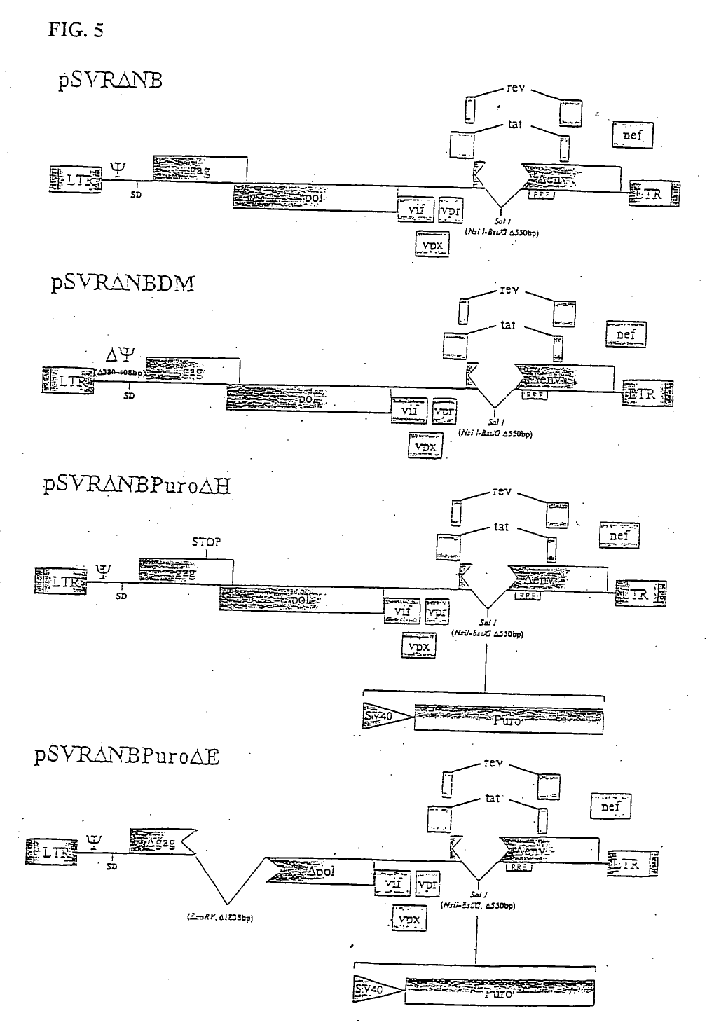

Novel HIV-2 vectors were designed containing the puromycin resistance selectable marker based on an env deleted HIV-2 provirus, pSVRΔNB (described below). Both vectors had the same deletion in env as the parental plasmid, and had the puro^{r} cassette at this locus. The first vector, pSVRΔNBPuroΔH, contained the same premature stop codon as pSVRΔH that was used in the RPA studies. The second vector, pSVRΔNBPuroΔE, contained a large deletion that removed the majority of the gag and pol ORFs. In order to assess competition effects, help was provided by the parental pSVRΔNB compared to pSVRΔNBDM, which contains the DM deletion. The structures of the constructs used are shown in (FIG. 5). Vectors were pseudotyped with the VSV-G glycoprotein and their ability to transduce HeLa CD4⁺ LTR-βgal cells assessed.

Concentrated supernatants were prepared from COS-1 transient transfections (described below) with 5 μg each of vector & helper plasmids, along with 2 μg of VSV-G expression construct, pCMV-VSVG (described below), or empty vector. Twelve well dishes containing of HeLa CD4⁺ LTR-βgal cells at 20% confluence were then transduced with COS-1 supernatants containing an equivalent amount of RT activity. Three days post-transduction, selection media containing puromycin was applied to the cells. Selection was maintained until all mock-transduced cells were dead. Puromycin resistance was not seen in envelope negative transduced control cells after selection, indicating that the ΔNB deletion is sufficient to abrogate function of the HIV-2 Env glycoprotein. Cells were fixed and stained as described, and the number of colonies counted and the results expressed as colony forming units per 10000 units of RT activity (cfa/100000 RTU).

The DM deleted construct was a far more efficient helper than the wild type. This is in accordance with the data from packaging described above. Furthermore, there was a difference between the vector that contained a stop mutation in gag compared to the vector with the deletion, the former giving far higher titres. In order to confirm that the differences in titre corresponded to differences in the packaging efficiencies of the vector, analogous COS-1 cell transfections were assessed by RPA. The relative packaging efficiencies of the vectors did, indeed, correspond to the vector titre, with the most efficient combination being a DM deleted helper packaging a vector without a large deletion in the gag ORF.

The results confirm that an HIV-2 helper containing an intact Ψ is unable to perform efficiently in vector systems, due to the co-translational packaging mechanism the virus employs. Competition for limiting Gag polyprotein, however, allows production of comparatively high titre vector preparations using a Ψ deleted helper.

### EXAMPLE 3

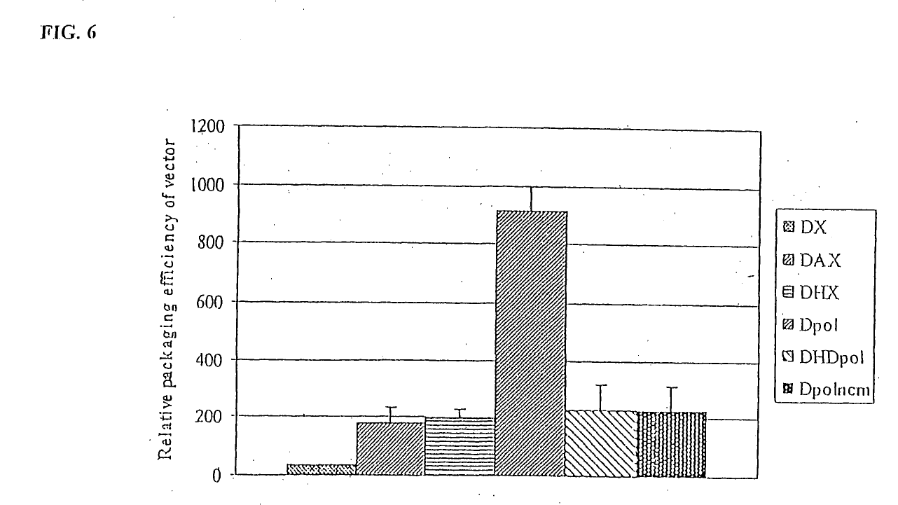

The differences in both titre and packaging efficiency between different puromycin resistant vectors might implicate cis-acting signals present in the gag ORF. No effect of including such regions when wild type HIV-2 packages vector RNAs has previously been observed (Kaye and Lever (1999), as above). The ability of a DM mutant virus to package a panel of HIV-2 vectors that contained differing lengths of the gag ORF was tested. All had the pol ORF deleted. Equal amounts (5 μg) of vector and pSVRDM were transfected into COS-1 cells, and RNA packaging assessed by RPA (FIG. 6).

As shown previously, the HIV-2 vector containing an intact gag ORF, pSVRΔpol, is capable of efficiently packaging its own RNA, and serves as a positive control. In contrast, pSVRΔHΔpol, that contains the premature stop codon is efficiently packaged by Gag provided by the DM mutant helper, albeit to a lesser extent. This construct contains the entire gag ORF, and so possesses any cis-acting signals contained therein. Ribosomal scanning of the entire gag ORF appears unnecessary for efficient vector packaging, as pSVRΔpolncm is packaged to a similar level as the previous construct. Removal of sequences up to, and including the 3′ region of MA also has no detrimental effect on vector packaging, as constructs pSVRΔHX and pSVRΔAX are both packaged to the same level as the above constructs. In contrast to the other vectors, pSVRΔX was packaged very poorly by the DM helper virus. This vector has a deletion of almost the entire gag ORF, starting from near the ATG (position 553). This indicates that there may be a signal in the 5′ part of gag, specifically in MA, that enhances packaging, or alternatively, that ribosomal scanning of this region may be important in promoting correct folding of RNA structures present in the leader or in gag itself. This might be a way in which translation and packaging are linked in the HIV-2 infected cell.

### EXAMPLE 4

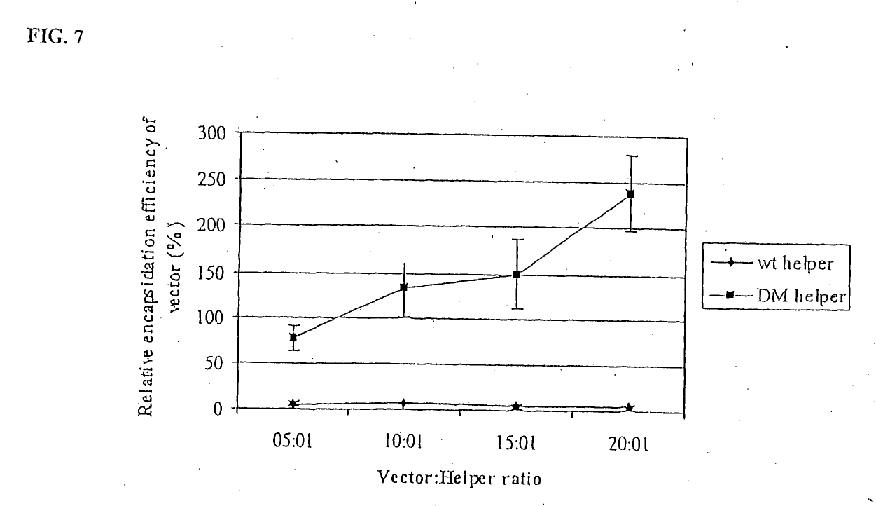

No single deletion in any lentiviral system completely abrogates packaging of viral RNA. This is probably due to the functional redundancy in packaging signals. Contamination of prospective therapeutic vector preparations with helper virus sequences is, therefore, a major bio-safety issue. To investigate whether the fact that Gag levels appear to be limiting might allow complete removal of helper RNAs by competition, COS-1 cells were co-transfected with increasing amounts of stop codon-containing vector, pSVRΔHΔpol, along with a fixed amount of either pSVR or pSVRDM and the effects on packaging by RPA analysed (FIG. 7). A compensatory amount of non-HIV-2 stuffer DNA, pBluescript KSII+, was transfected where necessary in order to bring the total DNA used to 21 μg in each case.

Vector was only efficiently packaged in trans by the DM deleted virus. Even at vector:helper ratios of 20:1, wild type HIV-2 does not efficiently package vector RNAs, indicating that the coupling of translation and packaging in HIV-2 is very strong indeed. In contrast the amount of vector packaging by pSVRDM increases slowly as the vector:helper ratio increases. In addition, the efficiency of vector packaging is reduced, even at 5:1, compared to when the two are transfected in equal amounts. This is due to there being an enormous amount of vector RNA present in the cytoplasm that is unable to be packaged by the limiting amounts of Gag present. Instead, the cause of the apparent increase in vector packaging is a reduction in the amount of pSVRDM RNA being packaged. The shift in virion:cytoplasmic RNA ratios between helper and vector, therefore, leads to an apparent increase in packaging efficiency of the vector. Although the levels of pSVRDM RNA in the virion fraction of these experiments is not reduced to zero, the levels are only just measurable above background, whereas wild type HIV-2 maintains a high level of packaging. These experiments indicate, therefore, that it would theoretically be possible to completely titrate out packaging of a DM deleted helper from HIV-2 vector preparations.

Methods:

Plasmid construction. pSVR is an infectious proviral clone of the ROD strain of HIV-2 containing the replication origin of simian virus 40 and has been described previously (McCann and Lever (1997)). Restriction sites, where given, are numbered relative to the first nucleotide of the spiral RNA. Proviral constructs pSVRΔ1, 2, 3, & 4 containing deletions in the 5′ leader region have been previously described. The positions of these and newly introduced deletions are shown (FIG. 2). Deletion mutations in the 5′ leader were introduced by site-directed mutagenesis following the Kunkel method (Kunkel et al. (1987)) into a subclone of HIV-2, pGRAXS that has been previously described (Kaye and Lever (1998)). The mutagenic oligonucleotide used for construction of the Ψ1 deletion was 5′-GGCAGCGTGGAGCGGGGTGAAGGTAAGTACC-3′ and for the DM deletion 5′-GGCAGTAAGGGCGGCAGGAGCGCGGGCCGAGGTACCAAAGGC-3′. Sequences from the resulting subclones, pGRAXΨ1 & pGRAXDM containing the deletions were then introduced into the provirus by exchanging an Aat II (position −1384)-Xho I (position 2032) fragment. The DM/Ψ1 double mutation was constructed by mutating pGRAXDM using the Ψ1 oligonucleotide and introducing this into the provirus.

The HIV-2 vector pSVRΔH is a vector based on pSVR containing a premature stop codon in the capsid (CA) region of the gag ORF. This was generated by digestion of a HindIII site (position 1458) and subsequent re-filling with the Klenow fragment of T4 DNA polymerase followed by re-ligation of the DNA. pSVRDMΔH contains the DM deletion in the leader region and the stop codon from pSVRΔH; it was generated by introducing an EcoRV position 1101)-XhoI (position 2032) fragment from pSVRΔH into pGRAXDM. The AatII (position 11444)-XhoI (position 2032) fragment from this plasmid was then used to replace the same region of pSVR. pSEX was generated by introducing an artificial XbaI site at position 553 by site directed mutagenesis, as described above, using the mutagenic oligo 5′-GGAGATGGGCTCTAGAAACTCCG-3′. Subsequent partial digest with XbaI allowed removal of almost the entire gag and pol ORFs (553-5067). The HIV-2 vectors pSVRΔAX pSVRΔHX, pSVRΔpol, pSVRΔHΔpol, & pSVRΔpolncm have been described previously (Kaye and Lever (1999)). Briefly, pSVRΔAX contains a deletion from AccI (position 912) and Xba I (position 5067). pSVRΔHX contains a deletion from HindIII (position 1458 to Xba I (position 5067). pSVRΔpol contains a deletion from Xho I (position 2032) to Xba I (position 5067). pSVRΔHΔpol was constructed by introducing a translation stop codon at the Hind III site (position 1458) of pSVRΔpol by filling in the 5′ overhanging ends of the Hind III site with Klenow polymerase and religating the blunt ends. pSVRΔNB was generated as follows; an EheI fragment (306-5864) was removed from pSVR to generate pSVRΔE. This was subsequently digested with NsiI & BstXI, deleting a 550 bp fragment of the env gene (6369-6919), but leaving the RRE, and also the rev and tat ORFs intact. A DNA linker containing a SalI site was ligated into this position after blunting with T4 DNA polymerase as described above, generating pSVRΔEnBSalI. The EheI fragment was then re-introduced into this plasmid giving pSVRΔNB (FIG. 5). pSVRΔNBDM (FIG. 5) was generated by replacing the AatII-XhoI (11444-2037) region of pSVRΔNB with the same region from pSVRDM. pSVRΔNBPuroΔE and pSVRΔNBPuroΔH (FIG. 5) are both based on pSVRΔNB, having had a SalI fragment from plasmid KSIISVPuro introduced into the linker site, and subsequent removal of an EcoRV fragment (1101-2939) or replacement of said fragment with the same region of pSVRΔH, respectively, pCMV-VSVG contains the VSV G glycoprotein gene in the context of pCDNA3 (Invitrogen). AD plasmids based on HIV-2 proviral sequences were grown in TOPF'10 (Invitrogen) E. coli at 30° C. or room temperature to avoid recombination. All other plasmids were grown in DH5α E. coli under standard conditions.

Plasmids used for generation of anti-sense riboprobes for use in RNase Protection Assays (RPAs) were generated as follows. Plasmids KS2ΨKE and KS2ES have been described previously (Katie and Lever (1998); Katie and Lever (1999)). They generate anti-sense transcripts to regions of the HIV-2 genome corresponding to positions 306-751 & 4915-5284 respectively, and are in the context of the Bluescript KSII+ transcription vector (Stratagene). Plasmid KS2ΨEP generates an anti-sense-probe to viral sequence between EheI (position 306) and PstI (position −286) and is also in the context of Bluescript. Plasmid SKH2CA generates an anti-sense probe to the CA region of the gag ORF. In vitro transcription of linearised template DNA was carried out using T3, or in the case of SKH2CA T7, RNA polymerase and the Riboprobe transcription system (Promega).

Cell culture and transfection. COS-1 Simian epithelioid cells were maintained in Dulbecco's modified Eagle's medium (Gibco BRL) supplemented with 10% foetal calf serum, penicillin and streptomycin. Cells were transfected in 10 cm diameter dishes by the DEAE Dextran method (Mortlock et al., 1993) with a total of 10 μg of DNA. Cells and supernatants were harvested 44-48 h later and virus production assessed by reverse transcriptase assay (Potts, 1990). Jurkat T-cells were maintained in RPMI-10 medium (Gibco BRL) supplemented with 10% foetal calf serum, penicillin, and streptomycin. HeLa CD4⁺ LTR-βgal cells were maintained in Dulbecco's modified Eagle's medium as described (Page et al., 1990).

Protein analysis. COS-1 cells were metabolically labelled with ³⁵S-methionine (>1000 Ci/mmol) (Amersham) from 44 to 48 hours post transfection. Viral proteins were harvested from cellular and virion fractions, and visualised as described previously (Kaye and Lever, 1999).

T-cell replication assay. 10 ml supernatants from transfected COS-1 cells were removed 48 h post-transfection and passed through a 0.45 μM filter into a tube containing 5 ml 30% polyethylene glycol 8000 in 0.4 M NaCl. The contents were mixed by inversion and left overnight at 4° C. The next day virions were pelleted by centrifugation at 2000 rpm in a bench-top centrifuge rotor at 4° C. for 40 min. The pellets were then resuspended in 0.5 ml TNE (10 ml Tris-Cl pH7.5, 150 mM NaCl, 1 mM EDTA pH7.5), and a 10 μl sample was taken to measure particle production by reverse transcriptase assay. The remainder was then layered over 0.5 ml of 20% sucrose in TNE. Virions were purified by centrifugation at 40000 rpm in a Beckman TLA-45 rotor at 4° C. for 2 h. Pelleted virus was re-suspended in 100 μl RPMI-10 media and an amount equivalent to 500 000 units of reverse transcriptase activity added to 50 000 Jurkat T-cells in one well of a U-bottom 96-well culture plate, in a final volume of 200 μl. Any given well only received virus from one transfection supernatant; virus was not pooled at any stage. Replication was followed every three to four days by reverse transcriptase assay. A 10 μl sample was removed from each well for the assay, along, with a further 40 l. Fresh media was then added to the original volume, fresh Jurkats were not added during the assay.

RNA isolation. Cytoplasmic and virion RNA was harvested, purified, DNase treated and stored as described previously (Kaye and Lever (1998); Kaye and Lever (1999)).

Ribonuclease Protection Assay (RPA). ³²P-labelled anti-sense riboprobes were transcribed in vitro from linearised DNA templates using the Riboprobe system (Promega) T3 or T7 RNA polymerase. Riboprobes were purified from 5% polyacrylamide-8 M urea gels prior to use.

Reagents for RPAs were obtained from a commercially available it (Ambion). RNA inputs were normalised for all reactions; for cytoplasmic RNA, sample concentration was determined by spectophotometry and the same amount included in each tube, typically 1 μg. Virion RNA input was normalised by reverse transcriptase activity, with 50 000 units equivalent being the standard amount used per reaction. RNA was co-precipitated with 2×10⁵ cpm of riboprobe and 3 μg of carrier RNA from Torrula Yeast (Ambion). Hybridisation, and subsequent nuclease protection was carried out according to manufacturers instructions. Pelleted RNA was resuspended in RNA loading buffer (Ambion), separated on a 5% polyacrylamide-8 M urea gel, visualised by autoradiography, and quantified using a real-time Instant Imager (Packard). Size determination of fragments was achieved by running ³²P-labelled RNA markers made using the Century Marker template set (Ambion) in parallel.

For each experiment, a separate RPA was performed using the same RNA inputs, but probing for viral plasmid DNA using a probe generated from plasmid KS2ΨEP. In addition, a probe to human β-actin RNA (Ambion) was included in the reaction to control for variations in cytoplasmic RNA input. Any DNA contamination or variations in the β-actin signal was accounted for when calculating packaging efficiencies; taken as the ratio of virion to cytoplasmic RNA of a mutant relative to that of wild type.

Transduction and selection of HeLa CD4⁺ LTR-βgal cells. Supernatants from COS-1 cells transfected with helper, vector, env-expressor or empty env-expressor backbone, as well as mock-transfected cells, was harvested as described above, except that the resulting pellet was resuspended in 100 μl of Dulbecco's modified Eagle's medium. The RT activity of the resulting vector preparations was then determined as above, and the amount added to a 12-well dish of cells normalised in this way. Each well was at 20% confluence at the time that vector was added. After three days, media was replaced with selection media containing the appropriate antibiotics for maintaining the cell line, as well as 1 μg/ml Puromycin. Cells were then maintained under selection until all in the mock-transduced wells were dead. The wells were then fixed and stained for β-galactosidase as described previously (Page et al., 1990), and the number of colonies counted in each well. Transduction efficiencies were expressed as colony, forming units per 10000 units of RT activity (cfu/10000 RTU).

1. A Human Immunodeficiency Virus type 2 (HIV-2) vector comprising a mutation within an HIV-2 packaging signal such that viral RNA is not packaged within an HIV-2 capsid, wherein the mutation comprises deletion of
(a) a sequence of SEQ ID no 1 or a variant thereof,
(b) an internal fragment thereof of 5 or more nucleotides in length, or
(c) a fragment thereof of 17 or more nucleotides in length.

2. A vector comprising an HIV-2 packaging signal sufficient to package the vector in the HIV-2 capsid and a heterologous gene capable of being expressed by the vector.

3. A vector according to claim 2 comprising
(a) a sequence of SEQ ID no 1 or a variant thereof,
(b) an internal fragment thereof of 5 or more nucleotides in length, or
(c) a fragment thereof of 17 or more nucleotides in length.

4. A vector according to claim 2 or 3 comprising the matrix (MA) region of the gag ORF or a fragment thereof.

5. A vector according to claim 2 or 3 comprising nucleic acids 553 to 912 of HIV-2 RNA or a fragment thereof.

6. A vector according to any one of claims 2 to 5 wherein the heterologous gene encodes a therapeutic protein or peptide, an antigen protein or peptide.

7. A process for producing an HIV-2 virus encoding a heterologous gene, which process comprises infecting a host cell with a vector which is capable of producing HIV-2 capsid and a vector according to any one of claims 2 to 6; and culturing the host cell.

8. A process according to claim 7 wherein the vector which is capable of producing HIV-2 capsid is a vector according to claim 1.

9. A virus produced by the method of claim 7 or 8.

10. A pharmaceutical composition comprising a virus according to claim 9 and a pharmaceutically acceptable carrier.


SEQUENCE LISTING (5 sequences)

SEQ ID NO: 1 - DNA, 28 bp, Human immunodeficiency virus type 2
aacaaaccacgacggagtgctcctagaa

SEQ ID NO: 2 - DNA, 31 bp, Artificial Sequence
  mutagenic oligonucleotide
ggcagcgtggagcggggtgaaggtaagtacc

SEQ ID NO: 3 - DNA, 42 bp, Artificial Sequence
  mutagenic oligonucleotide
ggcagtaagggcggcaggagcgcgggccgaggtaccaaaggc

SEQ ID NO: 4 - DNA, 23 bp, Artificial Sequence
  mutagenic oligonucleotide
ggagatgggctctagaaactccg

SEQ ID NO: 5 - RNA, 49 nt, Human immunodeficiency virus type 2
ugcuccuagaaaggcgcgggccgagguaccaaaggcagcguguggagcg